(19) United States
(12) Patent Application Publication
(10) Pub. No.: US 2007/0259022 A1
(43) Pub. Date: Nov. 8, 2007

(54) Composition Comprising Statin

(76) Inventors: Henricus Otto Franciscus Molhuizen, Vlaardingen (NL); Yvonne Susanna J Veldhuizen, Vlaardingen (NL); Rainer Theo Weisbecker, Heilbronn (DE)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 11/660,849

(22) PCT Filed: Aug. 1, 2005
(86) PCT No.: PCT/EP05/08380

§ 371 Date: Feb. 20, 2007

(30) Foreign Application Priority Data
Aug. 23, 2004 (EP) 04077387.1

(51) Int. Cl. A61K 47/00; A61K 31/351; A61P 9/10

(52) U.S. Cl. 424/439; 514/460

(57) ABSTRACT

Composition comprising at least 0.001 mg/g statin wherein the composition is a particulate comprising a matrix material in an amount of 10-70 wt % and fatty matter dispersed in the matrix material, wherein the amount of fatty matter and statin together is 30-90 wt % and wherein the matrix material comprises protein or a carbohydrate or a combination thereof. The particulate composition may suitably be used as a creamer. The invention also relates to processes for preparing compositions comprising statins and food products comprising the compositions.

### TECHNICAL FIELD

The present invention relates to a composition comprising statin. The invention further relates to a process for preparing such composition and the use of the composition. In addition, the invention relates to a food product comprising the composition of the present invention and a process of preparing such food product.

### BACKGROUND OF THE INVENTION

Cardiovascular disease is a leading cause of morbidity and mortality, particularly in the United States and in Western European countries and is emerging in developing countries. Several factors are mentioned in relation to the development of cardiovascular disease including hereditary predisposition to the disease, gender, lifestyle factors such as smoking and diet, age, hypertension, and hyperlipidemia, including hypercholesteremia. Several of these factors, particularly hyperlipidemia and hypercholesteremia, contribute to the development of atherosclerosis, a primary cause of vascular and heart disease.

The literature suggests that elevated low-density lipoprotein cholesterol (hereafter “LDL-cholesterol”) is related to an increased risk of coronary heart disease.

Statins are compounds that are known to have a lowering effect on levels of LDL-cholesterol in the human blood. Statins inhibit the hydroxymethylglutaryl coenzyme A (HMG-CoA) reductase, the rate-determining step in the cholesterol biosynthesis.

Scientific research has confirmed the healthy properties of statins especially with respect to LDL blood-cholesterol and triglyceride levels lowering activities, both in animals and in humans (Li et al., Nutrition Research 18, 71-81 (1998); Heber et al., Am. J. Clin. Nutr. 69, 231-236 (1999)).

The presence of statins in food consumed by humans is associated with a lower level of LDL-cholesterol and lower risk of coronary heart disease.

For the preparation of food containing statins, it is advantageous to have a statin source that has a high statin content and is widely and easily applicable in food products and is stable upon storage and transportation.

In WO02/063976, fermented soy material comprising statin is used for the preparation of the food products. This fermented soy material comprises complete soy beans. When this material is used for food products the typical soy flavour will also be incorporated into the food. Although some people like this taste, there are many people who do not like the taste of soy.

WO 02/10394 describes oral pharmaceutical formulations comprising a suspension of statin containing particles in liquid oil and a pharmaceutically acceptable carrier that is adapted for releasing the suspension in the gastric tract and/or the intestine tract. Typically, the carrier is a hard gelatine capsule.

WO 02/43659 describes compositions containing statins and calcium for improved cardiovascular health. The compositions according to the international patent application may contain about 125 to 500 mg docosahexaenoic acid (DHA). It is observed in the international application that the composition may take the form of a powder which may comprise an edible carbohydrate material such as lactose or starch.

It is an object of the present invention to provide a composition comprising statins that can be used for the manufacture of food products. It is another object of the present invention to provide a composition comprising statins that is easily applicable to many food products and also is stable upon storage and transportation and is not prone to deterioration.

We have now surprisingly found that one or more of the above objects is obtained by a composition comprising statin wherein the composition is a particulate comprising matrix material and fatty matter dispersed in said matrix material, which composition can be used as a creamer/whitener.

### SUMMARY OF THE INVENTION

Accordingly in a first aspect the invention relates to a composition comprising at least 0.001 mg/g statin wherein the composition is a particulate comprising a matrix material in an amount of 10-70 wt % and fatty matter dispersed in the matrix material, wherein the combined amount of fatty matter and statin is 30-90 wt %, and wherein the matrix material comprises protein or a carbohydrate or a combination thereof.

A second aspect of the invention relates to a food composition comprising the composition according to the first aspect of the invention.

A third aspect of the invention relates to a process for the manufacture of the composition according to the first aspect of the invention.

A fourth aspect of the invention relates to the use of the composition according to the first aspect of the invention as a creamer.

### DETAILED DESCRIPTION OF THE INVENTION

Many food products like e.g. cream-style soups and sauces, both wet and dry mixtures thereof, and instant dishes like pasta with a sauce, often contain an ingredient which is referred to as a creamer, whitener, or thickener. These products usually contain fat blends that can provide a creamy taste, mouthfeel, improved body, viscosity and/or a whitening effect. These creamers are also known for application in drinks such as tea and coffee including variants such as cappuccino, Wiener melange, café-au-lait, as cocoa drinks, milk shakes, (soy) milk, etcetera, also to provide a creaming and/or whitening effect. Often, but not always, such drinks are offered as dry, instant compositions. The creamer may be part of such composition, or may be offered separately for application in such drinks. Creamers usually comprise fat to provide a creamy taste and mouthfeel and other material such as carbohydrates and protein.

The present invention relates to a composition comprising at least 0.001 mg/g statin wherein the composition is a particulate comprising a matrix material in an amount of 10-70 wt % and fatty matter dispersed in the matrix material, wherein the combined amount of fatty matter and statin is 30-90 wt %, and wherein the matrix material comprises protein or a carbohydrate or a combination thereof. Preferably, the composition comprises 20-70 wt % matrix material and 30-80 wt % of fatty matter.

Particulate in this context is to be understood as powder, flakes, cubes, pellets etcetera (i.e. non fluid). In a preferred embodiment, the particulate is in the shape of flakes, granules, powder, cube, pellet, or tablet. More preferably, the particulate is in the shape of granules or powder.

In the particulates according to the invention the fatty matter is essentially dispersed in the matrix material, preferably as discrete regions. More preferably, the fatty matter is dispersed in the matrix material as oily or fatty matter droplets, crystals or particles. As an alternative or more specific embodiment, the fatty matter is preferably present as oily or fatty matter droplets or crystals which droplets or crystals are at least partly covered by or encapsulated with the matrix material.

In the context of the invention “essentially dispersed” means that at least 50% of the fatty matter is dispersed in the matrix material. This can be identified by using microscopic techniques. Preferably at least 60%, more preferred from 70 to 100 wt % of the fatty matter is dispersed in the matrix material.

The compositions according to the invention are preferably “dry” preparations. However, such compositions still may contain a considerable amount of water, e.g. as a result of an incomplete dehydration process or as a result of water naturally present in the constituents. The amount of moisture present in the compositions according to the invention is preferably below 20 wt %, most preferably below 10 wt %.

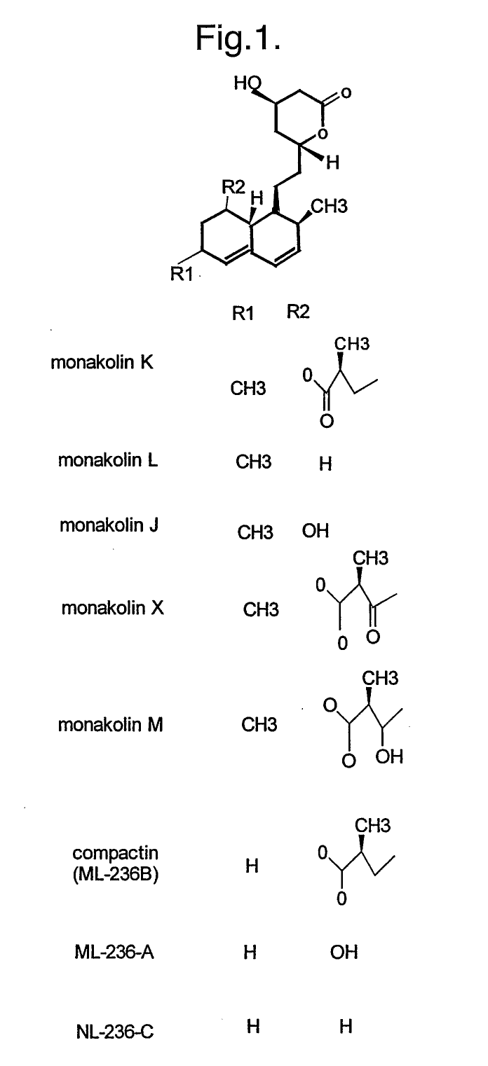

The composition according to the present invention comprises statins. Statins are defined as substances having the structural formula, presented in FIG. 1. In this structural formula, R1 and R2 can be any group. Preferred statins are those which are given in FIG. 1.

The amount of statins given below will be expressed, in wt % or weight parts per million (ppm), mg/kg or mg/g, relative to the total weight of the food product, unless otherwise indicated.

The amount of statins given herein are the sum of the amounts of individual statins, as e.g. determined by high performance liquid chromatography (HPLC) or LC-MS, unless otherwise indicated.

Preferably, the composition according to the present invention comprises 0.01 to 1 mg/g statin. Even more preferred the composition of the invention comprises at least 0.01 to 0.5 mg/g statin and even more preferred 0.04 to 0.2 mg/g statin.

The source of statins may be any available source. The statins may be synthetically or semi-synthetically produced. Preferably the statins are food grade, such as those produced by fermentation. Suitably the statin are produced by Monascus fungi grown on soya beans such as described in WO02/64809 and WO02/063976.

The present compositions advantageously comprises 15-70 wt. % of matrix material. More preferably the composition comprises 20-70 wt. %, most preferably 25-70 wt. % of matrix material.

To be suitable in these applications the fatty matter must have the appropriate physical properties in terms of melting behavior, crystallization behavior, brittleness, organoleptic properties, taste, as well as physical and chemical stability. The fatty matter may comprise conventional fat or fat blend, oil, interesterified or fractionated fat, hydrogenated fat, but also fat-replacers, such sucrose esters, and other fatty material like diglycerides, monoglycerides, sterol and sterol esters, as long as it behaves well like having a good taste, a good mouthfeel and good cooking behavior.

The fatty matter in the compositions described above usually comprise a considerable amount triglycerides of fatty acids (hereinafter for short: triglycerides). The fats used are usually mixtures of various triglycerides.

The triglycerides (which form part or all of the fatty matter) are usually obtained from vegetable sources. Preferably the triglycerides have a low saturated fatty acids contents. It may be preferred that such triglycerides have specific fatty acid composition. The fatty acid composition can be denoted as follows, H stands for fully saturated fatty acids with C16 and longer chains (e.g. C16, C18, C20, C22 and C24), U stands for mono- and poly-cis-unsaturated fatty acid of any suitable chain length. H3 are triglycerides with fully saturated fatty acids with C16 or longer chains and H2U are triglycerides of 2 saturated fatty acids of 16 or more carbon atoms and 1 cis-unsaturated fatty acid.

In the present invention at least 55% wt (preferably at least 65% wt. based on the triglycerides in the composition according to the invention) are H3 and/or H2U. In the composition according to the invention it is preferred that the amount of H3 is at least 15% wt based on the total amount of triglycerides in the composition according to the invention, preferably at least 20%. Likewise, it is preferred that the amount of H2U taken together is at least 40% wt based on the total amount triglycerides in the composition according to the invention. Apart from said amounts of H3 and H2U it can be preferred to use fats in the composition in a particular ratio. In this case, the ratio H3/H2U is preferably between 0.5 and 1.2. Regarding the basic fatty acid composition, it is preferred that the amount of H is between 60 and 75% wt based on total amount of triglycerides in the particulates according to the invention. Normally, only fatty acids are used with even number of carbon atoms. Similarly, it is preferred that the amount of U is between 20 and 45% wt based on total amount of triglycerides in the composition according to the invention. In the composition according to the invention the amount of palmitic fatty acid (saturated fatty acid with 16 carbon atoms; C16:0) in the triglycerides is preferably between 30 and 70%, more preferably between 40 and 60% wt based on the total amount of triglycerides in the particulates according to the invention.

In a preferred embodiment, the fatty matter further comprises 5-80% of one or more phytosterols. Phytosterols are also known for their blood cholesterol lowering activity. Phytosterols herein, also known as plant sterols or vegetable sterols can be classified in three groups, 4-desmethylsterols, 4-monomethylsterols and 4,4′-dimethylsterols. In oils they mainly exist as free sterols and sterol esters of fatty acids although sterol glucosides and acylated sterol glucosides are also present. There are three major phytosterols namely beta-sitosterol, stigmasterol and campesterol. Schematic drawings of the components meant are as given in “Influence of Processing on Sterols of Edible Vegetable Oils”, S. P. Kochhar; Prog. Lipid Res. 22: pp. 161-188. The respective 5a-saturated derivatives such as sitostanol, campestanol and ergostanol and their derivatives are also encompassed in the term phytosterol. Preferably the phytosterol is selected from the group comprising fatty acid ester of β-sitosterol, β-sitostanol, campesterol, campestanol, stigmasterol, brassicasterol, brassicastanol or a mixture thereof.

The phytosterols in this preferred embodiment may be esterified with a fatty acid. Preferably the sterols may be esterified with one or more C2-22 fatty acids. For the purpose of the invention the term C2-22 fatty acid refers to any molecule comprising a C2-22 main chain and at least one acid group. Although not preferred within the present context the C2-22 main chain may be partially substituted or side chains may be present. Preferably, however the C2-22 fatty acids are linear molecules comprising one or two acid group(s) as end group(s).

Most preferred are linear C8-22 fatty acids as occur in natural oils. Suitable examples of any such fatty acids are acetic acid, propionic acid, butyric acid, caproic acid, caprylic acid, capric acid. Other suitable acids are for example citric acid, lactic acid, oxalic acid and maleic acid. Most preferred are myristic acid, lauric acid, palmitic acid, stearic acid, arachidic acid, behenic acid, oleic acid, cetoleic acid, erucic acid, elaidic acid, linoleic acid and linolenic acid. When desired a mixture of fatty acids may be used for esterification of the sterols. For example, it is possible to use a naturally occurring fat or oil as a source of the fatty acid and to carry out the esterification via an interesterification reaction. Use of a natural source nearly always results in a mixture of fatty acids. In a particular embodiment, the fatty acid mixture contains a high amount (>50 wt %, preferably >70 wt %, further preferred >wt80%) of unsaturated fatty acids, such as monounsaturated fatty acids (MUFA) and/or polyunsaturated fatty acids (PUFA). PUFAs are generally regarded as healthy and have blood cholesterol lowering capacity. In addition, sterols esters prepared with such fatty acids and also the composition prepared with it will have an extra blood cholesterol lowering capacity. Preferably fatty acid mixtures of sunflower, safflower, rapeseed, linseed, olive oil, linola and/or soybean are used. These are typical sources of high PUFA and/or low SAFA. Suitable esterification conditions are for example described in WO 92/19640.

(Mixtures for) cream-style soups and sauces, but also other products such as instant dishes like pasta with a sauce or wet soups and sauces often contain an ingredient which is referred to as a creamer, and/or creamer/whitener, and/or creamer/thickener. These products usually contain fat blends that can provide a creamy taste and/or mouthfeel and/or improved body and/or viscosity and/or a whitening effect. Such products, which are herein after called creamers for brevity, can also be in the form of e.g. tablets. Creamers are also known for application in (compositions for preparing) drinks such as tea, coffee, including variants such as cappuccino, Wiener melange, café-au-lait, etcetera, cocoa drinks, milk shakes, (soy) milk, etcetera, also to provide a creaming and/or whitening effect. Often, but not always, such drinks are offered as dry, instant compositions. The creamer can be part of such composition, or may be offered separately for application in such drinks. To be suitable in these applications the fat blends in the creamer must have the appropriate physical properties in terms of melting behaviour, crystallisation behaviour, brittleness, organoleptic properties, taste, as well as physical and chemical stability.

In order to increase stability, shelf life and solubility, and to give proper creaming or whitening behavior the fatty matter is preferably dispersed in another material, e.g. hydrophilic film forming materials. The fatty matter is preferably present as oily or fatty matter droplets or crystals which droplets or crystals are at least partly covered by or encapsulated with the matrix material.

In such dispersions the individual fatty matter particles as well as clusters of fatty matter particles may at least be partially covered by or dispersed in the matrix material. The fatty matter should therefore also be suitable for being submitted to encapsulation and drying processes in order to form free flowing and highly dispersible products. The covering, encapsulation or matrix material often contributes to the properties of the creamer.

According to a preferred embodiment, the present invention relates to a composition wherein the fatty matter is dispersed in the matrix material as discrete regions, preferably as fatty matter droplets, crystals or particles. More preferred the fatty matter droplets, crystals or particles are at least partly covered by or encapsulated with the matrix material.

The composition of the present invention advantageously comprises 30-90 wt % of fatty matter, preferably 30-80 wt % and more preferably 40-75 wt % of fatty matter. Most preferred compositions comprise 50-70 wt % of fatty matter.

It is preferred that the matrix material comprises at least a protein or a carbohydrate, but preferably both a protein and a carbohydrate are present in the matrix material. They may be present in a ratio by weight of protein:carbohydrate of between 1:0.2 and 1:20.

Preferred proteins for the purpose of the invention are proteins selected from the group consisting of dairy protein, vegetable protein, gelatine, and mixtures thereof. Optionally the proteins are hydrolyzed before use. Suitable vegetable protein are soy protein. For the present invention dairy proteins (e.g. whey protein or caseinate) are most suitable.

Suitable carbohydrates in the present invention are carbohydrates selected from the group consisting of maltodextrin, sugar (incl. e.g. glucose, sucrose, fructose, lactose), sugar derivatives (e.g. polyols), starch (incl. starch hydrolysates such as maltodextrin), flour, chemically modified starch, physically modified starch, xanthan, guar, locust bean gum, alginate, pectin, carrageenan, polydextrose, and mixtures thereof. Suitable matrix material may be dairy products like liquid and/or powdered (skim) milk or cream.

In the present invention it is preferred that at least 60% by weight of the particulates has a size of 1-1000 μm, preferably 10-600 μm. It is also preferred that at least 60% by weight of the fatty matter droplets, crystals or particles has a size of 0.05-100 μm, preferably 0.1-20 μm.

Another aspect of the invention relates to a food product comprising 2-80 wt. % of an ingredient selected from the group consisting of salt, sugar, sweet starch hydrolysate, non-sugar sweetener and combinations thereof and 2-75 wt % of the particulate composition as defined herein before. The weight % is based on dry weight. In the present food product the particulate composition is usually identifiable as such, meaning that individual particles of the particulate composition are distinctly present in the food product.

The invention also relates to creamers and/or whiteners comprising 10-100% of the particulates according to the invention.

According to a preferred embodiment the present food product is an instant food product that can be reconstituted with water to obtain a reconstituted product with a pourable, preferably a liquid consistency. Typically, such an instant food product is provided in the form of a powder or a granulate. The water content of such instant food products typically does not exceed 20 wt. %, more preferably it does not exceed 10 wt. %.

One preferred embodiment relates to a food product comprising 2-50 wt % salt and 2-65 wt %, preferably 2-50 wt % of the composition according to the invention. Again, the weight % is based on dry weight. The food composition may further comprise 0-30 wt % mono-sodium-glutamate, 0-50 wt % fat and/or fatty matter, 0-20 wt % herbs and/or spices, 0-30 wt % vegetable particulates, and 0-30 wt % starch-based thickener.

Examples of such food products are soup- and sauce concentrates, which yield a soup or sauce upon dilution and heating with an aqueous liquid. The food products compositions may be in the form of flakes, granules, powder or agglomerated or e.g. pressed to a cube, pellet, or tablet. The food product may further comprise e.g. in an amount of 0.1-50 wt % vegetable powder, e.g. tomato powder.

In another embodiment the food product comprises 2-80 wt % preferably 20-70 wt % of an ingredient selected from the group consisting of sugar, sweet starch hydrolysate and non-sugar sweetener, 0.5-80 wt %, preferably 0.6-50 wt %, more preferably 0.8-20 wt % of:

tea powder, instant tea, tea flavour, tea colorant, dried tea, tea concentrate, coffee powder, instant coffee, coffee flavour, coffee colorant, dried coffee, coffee concentrate, cocoa powder, instant cocoa, cocoa flavour, cocoa colorant, dried cocoa, cocoa concentrate, milkshake powder, instant milkshake, milkshake flavour, milkshake colorant, dried milkshake, milkshake concentrate, milk powder, instant milk, milk flavour, milk colorant, dried milk, milk concentrate, soymilk powder, instant soymilk, soymilk flavour, soymilk colorant, dried soymilk, soymilk concentrate, dairy drink powder, instant dairy drink, dairy drink flavour, dairy drink colorant, dried dairy drink, dairy drink concentrate, fruit juice powder, instant fruit juice, fruit juice flavour, fruit juice colorant, dried fruit juice, fruit juice concentrate, fruit smoothie powder, instant fruit smoothie, fruit smoothie flavour, fruit smoothie colorant, dried fruit smoothie, and/or fruit smoothie concentrate, and further comprising 10-75 wt %, preferably 20-50 wt % of the composition according to the invention. The weight % is based on dry weight.

A preferred application is a creamy black tea or instant compositions for such. Thus, the invention further relates to a composition comprising 30-65 wt % sugar or sweet starch hydrolysate, or mixture thereof, 0.8-20 wt %, more preferred 1-10 wt % black tea powder and/or black tea flavour and further comprising 20-60% wt of the composition according to the invention. Preferably, the composition is an instant composition, which upon dilution with water and heating gives a creamy black tea.

The food product according to the invention preferably comprises statins in an amount sufficient to obtain a blood LDL-cholesterol lowering effect if the food product is used according to the common needs of the consumer.

Yet another preferred embodiment concerns the use of the aforementioned food products in manufacture of a medicament for the treatment or prevention of cardiovascular disease. The preferred intake of statin per day is herein 5-40 mg/day, more preferably 5-20 mg/day, even more preferably 8-15 mg/day. Furthermore, the intake of statin per day is preferably 1-5 mg/day, more preferably 1-2.5 mg/day.

The skilled person will be able to adjust the percentage of statins in the food product to obtain the desired blood cholesterol lowering effect. The percentages will depend on the type of food product, since the food products are used in different serving sizes. Moreover the pattern in a food product is consumed (servings per day and distribution over days) is dependent on the food product.

Preferably the food product comprises 0.1-10 mg statin per serving, more preferably 0.5-5 mg statin per serving and even more preferably 0.5-2.5 mg statin per serving.

Another embodiment of the present invention relates to a process for manufacturing the composition according to the invention, comprises the steps of a) preparing an emulsion or dispersion of the fatty matter and matrix material in an aqueous liquid, wherein at least part of the fatty matter is an oil comprising statin, and b) drying said emulsion or dispersion.

An oil comprising statin may suitably be obtained by extraction of a substrate which is fermented with a statin producing fungus. A preferred substrate is soybean. The extraction may appropriately be performed by super-critical CO₂.

A preferred embodiment relates to a process for manufacturing the composition according to the invention comprises the steps of a) preparing an emulsion or dispersion of the fatty matter and matrix material in an aqueous liquid, wherein at least part of the matrix material is a flour comprising statin, b) drying said emulsion or dispersion.

A flour comprising statin may be prepared by grinding a starchy plant material which is fermented by a statin producing fungus. The material is optionally defatted and dried.

The emulsion or dispersion of the fatty matter and matrix material in an aqueous liquid can be prepared by means as known in the art, e.g. high shear mixing (optionally followed by homogenizing), membrane emulsification techniques, or other means. The drying is preferably done by spray-drying but other drying processes such as for example heat drying (including vacuum freeze drying), air drying etc can also be employed.

Yet another embodiment of the present invention relates to the use of the composition according to the invention as a creamer or whitener. Apart from free flowing particulates such creamer and/or whitener can be in the form of e.g. a cube, pellet or tablet.

The creamer/whitener can be used as such e.g. for whitening tea or coffee or creaming soup and sauces, or the composition can be incorporated into food products such as tea, soups, sauces and concentrates thereof. In addition, the composition of the present invention can be used in the preparation of milk tea, soup, or sauce. The tea, soup or sauce may be in a dry format or may also be in a liquid format.

For example, the composition according to the invention may be applied in liquid or pasty products e.g. savoury products, in which a creaming effect is desired. Such liquid or pasty products usually contain some water, and when the composition according to the invention is incorporated in such liquid or pasty products the dry particulates will generally melt and/or dissolve, and no longer be visible as such. Examples of such liquid or pasty products are wet soups and sauces, which are often pasteurised or sterilised and aseptically packaged.

Hence, the present invention further relates to a process for preparing a liquid or pasty sauce, soup or concentrate of such a sauce or soup, which process includes the step of including 2-65 wt %, preferably 2-50 wt % of the composition according to the invention as set out herein in such liquid or pasty sauce, soup or concentrate of such a sauce or soup.

The composition according to the invention may also be used in applications for performing a creaming and/or whitening effect, such as beverages like tea, coffee, cocoa drinks, milk shakes, milk, soymilk, dairy drinks, fruit juices, fruit smoothies, etcetera, as well as compositions for preparing these. Often, such food products contain sugar or sweet or non-sweet starch hydrolysates or a non-sugar sweetener. The sugar, sweet or non-sweet starch hydrolysates or a non-sugar sweetener may be part of the creamer/whitener, or may be included in the formulation next to the creamer. In addition, such formulation may contain something to give the formulations its characteristic flavour and/or colour, such as extracts of—, flavourants and/or colorants for—, instant compositions for—, dried compositions of—: tea, coffee, cocoa, milk shakes, milk, soymilk, dairy drinks, fruit juices, and fruit smoothies, etcetera. Such applications may be offered to the consumer in a final (wet) form ready for consumption (optionally requiring heating or cooling), or in a dry form, e.g. as instant product, further requiring dilution with an aqueous liquid (e.g. water, milk) and optional heating and/or cooling or as a separate creamer/whitener offered for use in such beverages.

### EXAMPLES

General Procedures:

### Preparation of Oil Comprising Statin and Flour Comprising Statin

An oil comprising statins and flour comprising statins were prepared by supercritical extraction of ground fermented soybeans.

For the supercritical extraction of natural solid matrices, equipment and software of Thar Designs, USA was used. The CO₂ pump is capable of compressing liquid carbon dioxide to a pressure up to 600 bars at a constant flow-rate. In a static mixer, a polarity modifier may be mixed with the liquid carbon dioxide. In a pre-heater (not depicted) the carbon dioxide was heated to reach supercritical conditions before entering the extraction vessel.

In the extraction vessel, which was heated with a double wall heating mantle, the supercritical carbon dioxide was passed over the solid matrix for extraction.

Downstream of the extraction vessel, the supercritical carbon dioxide was expanded over an automated backpressure regulator. The backpressure regulator was coupled to a feedback control unit to control the pressure in the system. The carbon dioxide was separated from the extracted material (liquid/solid) in a cyclone separation system. The carbon dioxide left the cyclone at the top, while the extracted material remained in the cyclone. The liquids extracted from the solid matrix were recovered during the experiment by opening the valve at the bottom of the cyclone.

The carbon dioxide gas was further expanded over a further backpressure regulator, which was operated manually. A gas clock downstream of the backpressure regulator registers how much gas has been put through the system, before the carbon dioxide leaves the system at ambient pressure.

The process equipment is designed to operate at the following conditions:

Process Operation Conditions Supercritical Extraction Set-Up

| Parameter | Range |
|---|---|
| Flow rate of liquid carbondioxide | 5-150 g/min |
| Pressure | up to 600 bar |
| Temperature | 20-95° C. |
| Extractor volume | 500 mL |
| Cyclone volume | 200 mL |
| Matrix to be extracted | solid |

Soy beans were fermented with a statin producing fungus according to methods described in WO02/64809 and WO02/063976. The fermented soybeans were ground prior to extraction in a water-cooled universal mill (type M20, IKA, Germany) until a fine powder was obtained.

About 100 gr. of fermented and ground soybeans were put in the extraction vessel and the remaining volume was filled with small glass beads (2 mm diameter). The flow rate of liquid carbondioxide was 20 g·min⁻¹. In the attached cyclone separation vessel, the extracted oil was collected. The oil contains about 1 mg/g statin.

After the extraction, the extraction vessel was opened and a dry free-flowing soybean flour powder was obtained. The flour contained about 1 mg/g statin. The total extraction time was 2 hours.

### Example 1

### Creamer I

| amount wt | Ingredients: |
|---|---|
| 35% | Fat-Phase (Palm fat, fractionated) |
| 20% | Statin-enriched soy oil (1 mg/g statin) |
| 13% | Natrium Caseinate (Milk protein) |
| 10% | Lactose |
| 20% | Maltodextrin |
| 2% | Di sodium Phosphate |

Creamer Preparation

The fatty fractions were prepared by mixing the required amounts of statin-enriched soy oil into the fat-phase and heating up 60° C. in a blending vessel under nitrogen atmosphere for 10 min.

5 kg water and 2 kg of the ingredients in the relative amounts as set out in the table above are mixed in a mixing tank with an Ultraturrax for 5 min. at 55° C. and then homogenized in a homogenizer (Schroeder) at one stage, at 200 bar. The resulting suspension then was spray dried in a multi stage spray dryer (Niro). The inlet temperature was about 165° C.; the outlet temperature about 62° C. The dry particulate creamer was agglomerated for 5 minutes in an agglomeration process step (Glatt Agglomator, inlet temperature 80° C., outlet temperature 50° C.). The spray dried and agglomerated creamer was stored under cool conditions below 20° C. In the creamer, most of the fatty matter was dispersed in the matrix material. This was confirmed by light microscopy.

### Example 2

### Creamer II

| amount (%) | ingredient |
|---|---|
| 54.8% | Fat-phase (soy oil hardened, 44-46) |
| 0.2% | Statin-enriched soy oil (1 mg/g statin) |
| 13% | Natrium Caseinate (Milk protein) |
| 10% | Lactose |
| 20% | Maltodextrin |
| 2% | Di sodium Phosphate |

Creamer Preparation

5 kg water and 2 kg of the ingredients in the relative amounts as set out in the table above are processed according to example 1.

### Example 3

### Creamer III

| amount wt | Ingredients: |
|---|---|
| 45% | Fat-phase (soy oil, hardened 44-46) |
| 20% | Statin-enriched soy oil (1 mg/g statin) |
| 7% | Soy protein (veg. protein) |
| 17% | Glucose sirup |
| 9% | Maltodextrin |
| 1% | Lecithin (soya based) |
| 1% | Di sodium Phosphate |

Creamer Preparation

5 kg water and 2 kg of the ingredients in the relative amounts as set out in the table above are processed according to example 1.

### Example 4

### Creamer IV

| amount wt | Ingredients: |
|---|---|
| 34% | Fat-phase (rape seed oil) |
| 20% | Statin-enriched soy oil (1 mg/g statin) |
| 10% | Natrium Caseinate (Milk protein) |
| 10% | Lactose |
| 23% | Maltodextrin |
| 2.5% | Di sodium Phosphate |
| 0.5% | Tocopherols |

Creamer Preparation

5 kg water and 2 kg of the ingredients in the relative amounts as set out in the table above are processed according to example 1.

### Example 5

### Leek Cream-Style Sauce

A dry sauce mixture for a leek cream-style sauce was made by mixing:

| | |
|---|---|
| Creamer I | 28.40% |
| Heat/moisture-treated starch, dried | 14.76% |
| Leek powder | 36.63% |
| Xanthan | 1.05% |
| Common salt | 4.22% |
| Citric acid granular | 0.40% |
| Powdered onion and leek | 5.18% |
| Sugar | 1.10% |
| Various flavourings | 8.26% |

To prepare the creamy tomato sauce 40 g of this dry mixture can be stirred into 200 ml cold water, mixed and briefly boiled. About 2 mg of statins are present in the finished product per serving of 200 ml.

### Example 6

### Saffron Cream Soup

A dry soup mix for a saffron cream soup can be made by mixing:

| | |
|---|---|
| Creamer I | 32.94% |
| Heat/moisture-treated starch, dried | 15.73% |
| Skim milk powder | 21.32% |
| Xanthan | 1.12% |
| Common salt | 4.51% |
| Citric acid granular | 0.22% |
| Powdered onion and leek | 5.55% |
| Sugar | 2.50% |
| Saffron powder | 0.08% |
| Various flavourings | 16.03% |

To prepare the creamy saffron soup 40 g of this dry mixture can be stirred into 200 ml cold water, mixed and briefly brought to the boil. This soup then provides about 2.5 mg of statins per serving of 200 ml.

### Example 7

### Cream-Style Sauce

A dry sauce mixture for a cream-style sauce can be made by mixing:

| | |
|---|---|
| Creamer I | 26.00% |
| Waxy corn starch, dried | 25.91% |
| Lactose | 7.97% |
| Common salt | 8.09% |
| Roux white | 14.39% |
| Champignon extract powder | 2.77% |
| Powdered onion | 5.18% |
| Sugar | 1.43% |
| Various flavourings | 8.26% |

To prepare the creamy sauce 40 g of this dry mixture is stirred into 200 ml cold water, mixed and briefly brought to the boil. The creamy sauce then provides about 2 mg of statins per serving of 200 ml.

### Example 8

### Mushroom Cream Soup

A dry soup mix for a mushroom cream soup can be made by mixing:

| | |
|---|---|
| Creamer I | 28.40% |
| Heat/moisture-treated starch, dried | 14.76% |
| Skim milk powder | 22.14% |
| Xanthan | 1.05% |
| Common salt | 4.22% |
| Citric acid granular | 0.40% |
| Powdered onion and leek | 5.18% |
| Sugar | 1.10% |
| Powdered mushrooms and ceps | 14.49% |
| Various flavourings | 8.26% |

To prepare the creamy mushroom soup 40 g of this dry mixture can be stirred into 200 ml cold water, mixed and briefly boiled. The soup will provide about 2 mg of statins per serving of 200 ml.

### Example 9

### Tomato Cream Sauce

A dry sauce mixture for a creamy tomato sauce can be made by mixing:

| | |
|---|---|
| Creamer I | 28.40% |
| Heat/moisture-treated starch, dried | 14.76% |
| Tomato powder | 36.63% |
| Xanthan | 1.05% |
| Common salt | 4.22% |
| Citric acid granular | 0.40% |
| Powdered onion and leek | 5.18% |
| Sugar | 1.10% |
| Various flavourings | 8.26% |

To prepare the creamy tomato sauce 40 g of this dry mixture can be stirred into 200 ml cold water, mixed and briefly boiled. The tomato sauce will provide about 2 mg statins per serving of 200 ml.

### Example 10

### Cream Milk Tea

An instant composition for a sweet creamy milk tea can be made by mixing:

| | |
|---|---|
| Creamer I | 42% |
| Sugar | 52% |
| Black tea powder | 5% |
| Black tea flavour | 1% |

To prepare the sweet creamy milk tea 16.5 g of this dry mixture can be stirred into 150 ml hot water and mixed. The creamy milk tea then provides about 1 mg statins per serving of 200 ml

Instead of creamer I the other creamers can also be used in the above mentioned examples.

1. Composition comprising at least 0.001 mg/g, preferably 0.01-1 mg/g statin wherein the composition is a particulate comprising a matrix material in an amount of 10-70 wt % and fatty matter dispersed in the matrix material, wherein the combined amount of fatty matter and statin is 30-90 wt %, and wherein the matrix material comprises protein or a carbohydrate or a combination thereof.

2. Composition according to claim 1, wherein the fatty matter is dispersed in the matrix material, preferably as discrete regions.

3. Composition according to claim 1, wherein the fatty matter is dispersed in the matrix material as oily or fatty matter droplets, crystals or particles.

4. Composition according to claim 1, wherein the fatty matter is present as oily or fatty matter droplets or crystals wherein the droplets or crystals are at least partly covered by or encapsulated with the matrix material.

5. Composition according to claim 1, wherein at least 60 wt. % of the dispersed fatty matter has a size of 0.05-100 μm.

6. Composition according to claim 1, wherein the matrix material comprises protein and carbohydrate in a weight ratio of 1:0.2 to 1:20.

7. Composition according to claim 1, wherein the protein is selected from the group consisting of dairy protein, vegetable protein, gelatine, and mixtures thereof.

8. Composition according to claim 1, wherein the carbohydrate comprises an ingredient selected from the group consisting of maltodextrin, sugar, sugar derivative, starch, flour, chemically modified starch, physically modified starch, xanthan, guar, locust bean gum, alginate, pectin, carrageenan, polydextrose, and mixtures thereof.

9. Composition according to claim 1, wherein at least 60% by weight of the particulate has a size of 1-1000 μm, preferably of 10-600 μm.

10. Creamer and/or whitener comprising 10-100% of the composition according to claim 1 wherein the water content does not exceed 20 wt %.

11. Food composition comprising 2-80 wt. % of an ingredient selected from the group consisting of salt, sugar, sweet starch hydrolysate, non-sugar sweetener and combinations thereof and 2-75 wt % of the composition according to claim 1, wherein the water content does not exceed 20 wt %.

12. Food composition according to claim 11, comprising 2-50 wt % salt and 2-65 wt % of the particulate composition, wherein the water content does not exceed 20 wt %.

13. Food composition comprising 2-80 wt % of an ingredient selected from the group consisting of sugar, sweet starch hydrolysate and non-sugar sweetener, 0.5-80 wt % of:
a) tea powder, instant tea, tea flavour, tea colorant, dried tea, tea concentrate and/or
b) coffee powder, instant coffee, coffee flavour, coffee colorant, dried coffee, coffee concentrate and/or
c) cocoa powder, instant cocoa, cocoa flavour, cocoa colorant, dried cocoa, cocoa concentrate and/or
d) milkshake powder, instant milkshake, milkshake flavour, milkshake colorant, dried milkshake, milkshake concentrate and/or
e) milk powder, instant milk, milk flavour, milk colorant, dried milk, milk concentrate and/or
f) soymilk powder, instant soymilk, soymilk flavour, soymilk colorant, dried soymilk, soymilk concentrate and/or
g) dairy drink powder, instant dairy drink, dairy drink flavour, dairy drink colorant, dried dairy drink, dairy drink concentrate and/or
h) fruit juice powder, instant fruit juice, fruit juice flavour, fruit juice colorant, dried fruit juice, fruit juice concentrate and/or
i) fruit smoothie powder, instant fruit smoothie, fruit smoothie flavour, fruit smoothie colorant, dried fruit smoothie, and/or fruit smoothie concentrate
and further comprising 10-75 wt % of the particulate composition according to claim 1.

14. Food composition according to claim 13, comprising 30-65 wt % sugar or sweet starch hydrolysate or mixture thereof, 0.8-20 wt % black tea powder and/or black tea flavour and further comprising 20-60 wt % of the particulate composition.

15. Process for manufacturing the composition according to claim 1 comprising the steps of:
a) preparing an emulsion or dispersion by combining fatty matter, matrix material and an aqueous liquid, wherein at least part of the fatty matter is an oil comprising statin,
b) drying said emulsion or dispersion.

16. Process for manufacturing the composition according to claim 1, comprising the steps of:
a) preparing an emulsion or dispersion by combining fatty matter and matrix material in an aqueous liquid, wherein at least part of the matrix material is a flour comprising statin,
b) drying said emulsion or dispersion.

17. Use of the composition according to claim 1 as a creamer or whitener.

18. Use according to claim 17 in the preparation of milk tea, soup, or sauce.
